(19) United States
(12) Patent Application Publication
(10) Pub. No.: US 2017/0231533 A1
(43) Pub. Date: Aug. 17, 2017

(54) SYSTEMS AND METHODS FOR JOINT ACTIVITY MONITORING

(71) Applicant: THE TRUSTEES OF THE UNIVERSITY OF PENNSYLVANIA, Philadelphia, PA (US)

(72) Inventors: Feini Qu, Philadelphia, PA (US); Peter Gebhard, Philadelphia, PA (US)

(21) Appl. No.: 15/438,440

(22) Filed: Feb. 21, 2017

Related U.S. Application Data
(63) Continuation-in-part of application No. PCT/US2015/046337, filed on Aug. 21, 2015.
(60) Provisional application No. 62/040,591, filed on Aug. 22, 2014.

(51) Int. Cl. A61B 5/11; A61B 5/00; G01C 19/00; G01P 15/105; G01C 22/00; G01D 5/20

(52) U.S. Cl. CPC A61B 5/1126; G01C 22/006; G01D 5/2033; G01C 19/00; G01P 15/105; A61B 5/0004; A61B 5/112; A61B 5/7267; A61B 5/7282; A61B 5/7246; A61B 2562/0219; A61B 2562/0223; A61B 2503/40; A61B 2560/0223; A61B 2560/0214; A61B 2560/0475

(57) ABSTRACT

Joint analysis system for analyzing kinematics of an anatomical including a sensor device, a storage device, a magnet, and an analysis engine. The sensor device can be configured to be disposed on a first side of the joint and can have one or more sensors, a processor coupled to the one or more sensors, a wireless data transmitter coupled to the processor, a data storage device coupled to the processor, and a battery coupled to the sensors, processor, wireless data transmitter, and data storage device. The magnet can be configured to be disposed on the second side of the joint. The analysis engine can be configured to receive data from the sensors.

### CROSS-REFERENCE TO RELATED APPLICATIONS

This application is a continuation-in-part of International Application Serial No. PCT/US2015/046337, filed on Aug. 21, 2015, which claims priority to U.S. Provisional Application Ser. No. 62/040,591 filed on Aug. 22, 2014, each of which are incorporated by reference in their entireties herein and from which priority is claimed.

### BACKGROUND

Information regarding joint activity, for example, kinematics of joints, can be useful for a variety of uses. For example, physicians and physical therapists can monitor patient recovery time from orthopaedic injuries, track rehabilitation progress over time, and facilitate early detection of surgical complications. Additionally, objective quantification of joint function can be used for evaluating experimental treatments in translational models. Existing wearable animal monitors can use accelerometer-based sensors that can measure activity intensity. Existing devices that are capable of gait analysis can employ multiple sensors, can depend on species-dependent algorithms, and can be expensive.

Therefore, there is a need for a low-cost, all-in-one device that can provide information on joint kinematics in addition to basic activity level using a single sensor board.

### SUMMARY

The presently disclosed subject matter provides systems and methods for analyzing kinematics of an anatomical joint. The joint can have a first side and second side.

According to one aspect of the disclosed subject matter, systems for analyzing kinematics of a joint are provided. In an exemplary embodiment, the joint analysis can include a sensor device, a storage device, a magnet, and an analysis engine. The sensor device can be configured to be disposed on a first side of the joint and can include one or more sensors, a processor coupled to the one or more sensors, a wireless data transmitter coupled to the processor, a data storage device coupled to the processor, and a battery coupled to the sensors, processor, wireless data transmitter, and data storage device. The magnet can be configured to be disposed on the second side of the joint. The analysis engine can be configured to receive data from the sensors.

In some embodiments, the one or more sensors can include a magnetometer. The magnetometer sensor can be adapted to provide readings that are influenced by the magnetic field provided by the magnet to provide kinematic information of the joint. The one or more sensors can include an accelerometer. The one or more sensors can include a gyroscope.

In some embodiments, the one or more sensors can be configured to sense stride length. The one or more sensors can be configured to sense swing time. The one or more sensors can be configured to sense stance time. The one or more sensors can be configured to sense ambulation speed. The one or more sensors can be configured to sense distance traveled. The one or more sensors can be configured to sense gait symmetry. The one or more sensors can be configured to sense gait cadence. The one or more sensors can be configured to sense joint kinematics. The one or more sensors can be configured to sense a disrupted pattern of ambulation. The data analysis engine can be configured to recognize an abnormal gait or behavior.

In some embodiments, the system can include a base station. The base station can include a processor, a data storage device coupled to the processor, a user interface coupled to the processor, and a wireless data transmitter coupled to the processor and configured to communicate with the wireless data transmitter of the sensor device. The base station can include a display. The sensor device and magnet can be configured to be worn by an animal or human. The sensor device and the magnet can be configured to be implanted in an animal or human.

In another exemplary embodiment of the disclosed subject matter, methods to analyze kinematics of a joint of an animal are provided. An example method can include calibrating one or more sensors and a magnet relative the joint. The method can include sensing a magnetic field with the calibrated sensors while the joint exhibits motion and angular velocity to generate a signal. The method can include filtering signal noise, if any, from the signal and identifying joint motion based on the corresponding acceleration and angular velocity. The method can include calculating joint and gait kinematic parameters from the identified joint motion.

The description herein merely illustrates the principles of the disclosed subject matter. Various modifications and alteration to the described embodiments will be apparent to those skilled in the art in view of the teachings herein. Accordingly, the disclosure herein is intended to be illustrative, but not limiting, of the scope of the disclosed subject matter.

### BRIEF DESCRIPTION OF THE DRAWINGS

FIG. 1 illustrates a component diagram of an exemplary system in accordance with the disclosed subject matter.

FIG. 2 illustrates a diagram of how an exemplary system of the disclosed subject matter can be used on a human knee.

FIGS. 3A and 3B show exemplary sensor devices in accordance with the disclosed subject matter.

FIGS. 4A-4D provide data that can be used to calibrate the sensors.

FIGS. 5A-5C show exemplary data related to joint angle and angular velocity of a human knee.

FIG. 6 shows an exemplary method for detecting joint angle in an animal model.

FIGS. 7A-7C show exemplary data related to joint angle of a pig stifle joint (pig knee).

FIG. 8 shows example data from detecting short-term activity results in an animal model.

FIG. 9 shows example data from detecting long-term activity results in an animal model.

FIG. 10 shows a flow chart of an exemplary method of analyzing kinematics of a joint of an animal according to the disclosed subject matter.

FIGS. 11A-11D show a magnet based system for quantifying joint kinematics in accordance with the disclosed subject matter. FIG. 11A shows an experimental schematic.

FIG. 11B shows device hardware (scale=1 cm). FIG. 11C shows Average knee flexion angle measured by a joint activity monitoring device and a motion capture system. FIG. 11D shows angular velocity of the tibia during a normal human gait cycle (n=3).

FIGS. 12A and 12B provide data illustrating unsupervised activity monitoring demonstrates time course of recovery in a porcine model. FIG. 12A shows % activity pre- and post-surgery (n=4). FIG. 12B shows Activity normalized to the average baseline value (dashed line) over 10 weeks. *p≦0.05 vs. Baseline.

FIGS. 13A and 13B show altered joint kinematics during unprovoked ambulation post-surgery. FIG. 13A shows change in magnetic field over time. FIG. 13B shows angular velocity of the porcine tibia during a gait cycle pre- and post-surgery (n=8-15 steps/time point).

### DESCRIPTION

The methods and systems presented herein can be used for remotely monitoring anatomical joint and gait kinematics, as well as general activity. As used herein, “anatomical joint” can refer to joints of animals or humans that are locations where bones connect and are configured to allow movement. For example and not limitation, anatomical joints can include the human knee, human elbow, and pig stifle joint (pig knee), and other joints. The disclosed subject matter can help physicians and physical therapists monitor patient recovery from orthopaedic injuries, track rehabilitation progress over time, and facilitate early detection of surgical complications. Through remote monitoring and continuous, long-term data collection, physicians and physical therapists can detect trends during the patient's recovery process. The data can be used to highlight a need to adjust treatment based on recovery level and rate, identify additional rehabilitation exercises with faster recovery or revision surgery with slow recovery. Athletes, coaches and trainers can use the disclosed subject matter to characterize joint and gait kinematics during training. Such data can be used to enhance performance and prevent injury. When worn by the research subject, the device can wirelessly transmit data on acceleration, angular velocity, and magnetic field in 3D space, and can allow for remote, real time visualization and analysis of unprovoked and unsupervised activity. Additionally, the range of motion and frequency of joint flexion and extension can be derived by attaching a magnet distal to the articulating joint of interest and measuring changes in magnetic field strength. This can allow for a species-independent, individual assessment of joint kinematics using a single sensor. The device can facilitate the monitoring of pathological progression and therapeutic efficacy for animal and human subjects in orthopaedic research.

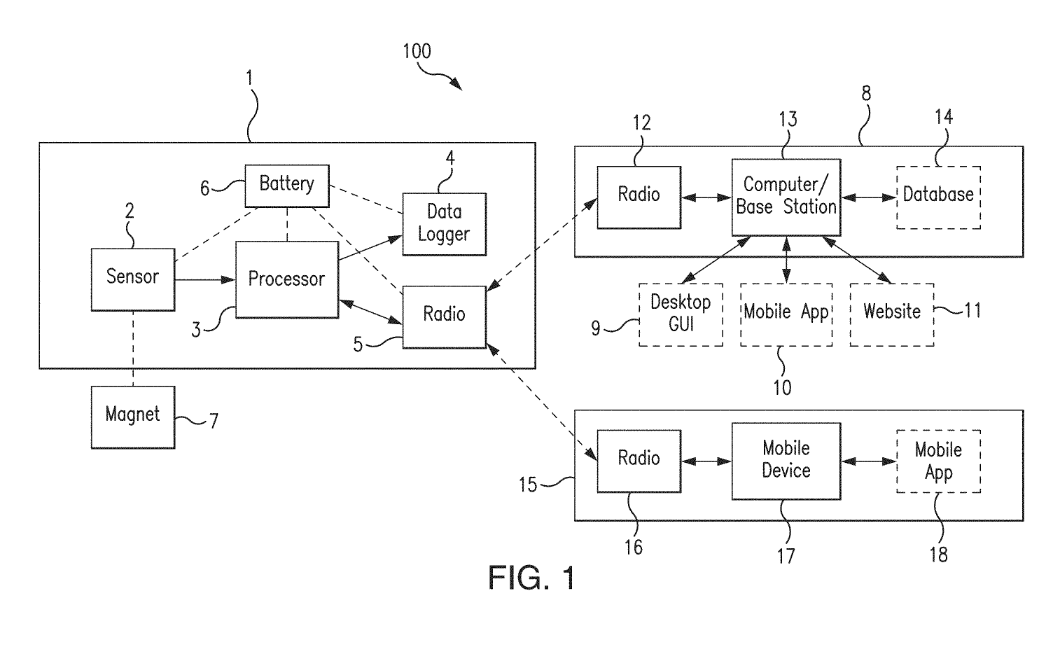

FIG. 1 shows, for the purpose of illustration and not limitation, a component diagram of an exemplary system 100 of the disclosed subject matter. The sensor device 1 can include a sensor 2, a processor 3, a data logger, 4, and a radio 5. The sensor 2 can integrate a triple-axis magnetometer, triple-axis accelerometer, and/or a triple-axis gyroscope. The sensor device 1 can also include a battery 6. The device can be designed to sense the magnetic field produced by a nearby magnet 7 (or other magnetic field producing object).

The system 100 can also include a base station 8 which can process and store outputs from the sensor device 1, and can display it over a variety of possible interfaces, for example, a desktop graphical user interface (GUI) 9, a mobile application 10 of a mobile communication device, such as a phone, tablet, laptop computer, or personal digital assistance (PDA), or a website 11, which can be accessed by a device with access to the internet. The base station 8 can include a radio 12 which can be configured to communicate with the radio 5 of the sensor device 1. The base station 8 can also include a computer 13 (for example, a processor) and a database 14 for storing information. The system 100 can also include a mobile device 15, such as a phone, tablet, laptop computer, or personal digital assistance (PDA), which can be used to receive data from the sensor device 1 and display the data. The mobile device 15 can have a radio 16 which can be configured to communicate with the radio 5 of the sensor device 1. The mobile device 15 can include a processor 17 which can have a mobile application 18 installed thereon. The data can be minimally processed and temporarily stored on the mobile device 15, but can be reviewed by a non-expert user. For example, the data displayed on the mobile device 15 can be displayed in a clear, intuitive way via a mobile app interface.

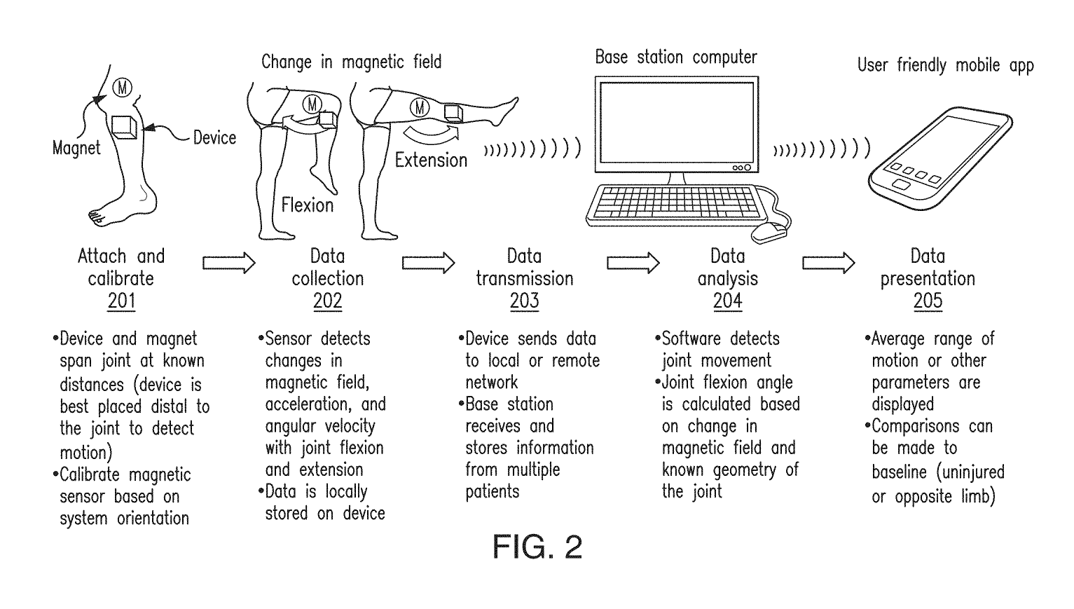

FIG. 2 shows, for the purpose of illustration and not limitation, an exemplary diagram of how an exemplary system can be used on a human knee. At 201, the device and magnet can be placed to span the joint, for example, a knee, at known distances. The device can be placed distal to the joint to detect motion and the magnet can be placed proximal to the joint. In some embodiments, the device can be placed proximal to the joint and the magnet can be placed distal to the joint. The magnetic sensor on the device can be calibrated based on the system orientation and relative positions of the device and magnet. At 202 data can be collected. The sensor can detect changes in the magnetic field, acceleration, and angular velocity with joint flexion and extension. Data can be locally stored on the device. At 203 data can be transmitted. The device can send data to a local or remote network. The base station can receive and store the information for multiple patients. At 204 the data can be analyzed. The software can detect joint movement. Joint flexion angle can be calculated based on change in magnetic field and known geometry of the joint. At 205 the data can be presented. The average range of motion or other parameters can be displayed on a user friendly mobile app. Comparisons can be made to baseline, for example, uninjured or opposite limbs.

The joint measurements can be visualized in real-time, and can reduce the time spent measuring range of motion with a conventional goniometer in a clinic or therapists office. By integrating the device into orthopaedic braces or clothing, the device can be incorporated into a user's daily activities. Real-time feedback displayed by a companion mobile application can help guide a patient's recovery. The guidance provided through the application can be automated based on the application's analysis of device feedback or can be managed by the patient's surgeon and/or physical therapist. The platform can promote patient compliance and encourage goal-oriented behavior.

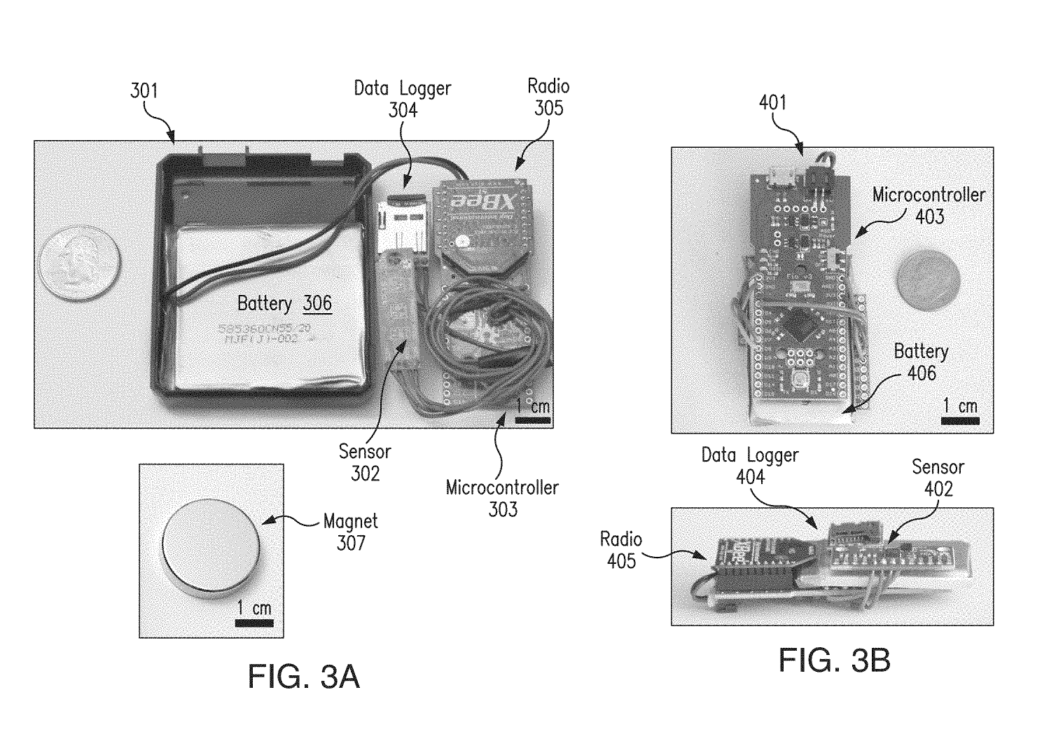

FIG. 3A shows an exemplary sensor device 301 having a battery 306, data logger 304, radio 305, sensor 302, and micro-controller (processor) 303. An exemplary magnet 307 is also provided. The black case of FIG. 3A can be made of plastic. The case can have dimensions of 7.5 cm×6 cm×2.5 cm and can weigh 27 g. The battery 306 can have a capacity of 2000 milliamp-hour (mAh), an energy rating of 7.4 watt-hour (Wh), dimensions that are 5.5 cm×5 cm×0.5 cm, and weigh 39 g. The sensor 302 can have dimensions of 3.5 cm×1 cm×0.3 cm and weigh 2 g. The micro-controller 303 can have dimensions of 6.5 cm×2.7 cm×1.5 cm and weigh 7 g. The radio 305 can dimensions of 2.7 cm×2.5 cm×0.7 cm and weigh 4 g. The data logger 304 can have dimensions of 1.9 cm×1.5 cm×0.3 cm and weigh 2 g. The entire device of FIG. 3A can weigh 81 g. FIG. 3B shows a top and side view of an exemplary sensor device 401 having a battery 406, data logger 404, radio 405, sensor 402, and micro-controller (processor) 403. The micro-controller 403, data logger 404, and radio 405 can have the same dimensions and weight as the corresponding elements in FIG. 3A. The sensor 402 can dimensions of 3.3 cm×1.5 cm×0.3 cm and weigh 2 g. The battery 406 can have a capacity of 850 mAh, a power rating of 3.1 Wh, dimensions of 4.8 cm×3 cm×0.4 cm, and weigh 16 g. The device 401 can have dimensions of 7 cm×3.5 cm×1.5 cm and can weigh 31 g. The circuit boards shown in FIGS. 3 A and B can be standard multi-layer printed circuit boards built using FR-4 glass-reinforced epoxy. The wiring can be basic hook-up wire, 22 American wire gauge (AWG). The batteries 306 and 406 can be, for example, 3.7 V polymer lithium ion batteries. A quarters and a dime are provided in FIGS. 3 A and B, respectively, to illustrate size.

The devices illustrated in FIGS. 3 A and B can be light-weight, palm-sized devices capable of measuring acceleration, angular velocity, and magnetic field strength in three dimensions. The device can be developed with off-the-shelf components at low cost. The sensor board can integrate a triple-axis accelerometer, a triple-axis gyroscope, and a triple-axis magnetometer, and can be calibrated prior to use via a custom software tool. A computer with a radio peripheral can receive transmitted data to plot it in real time.

The magnet used with the system can be a rare-earth neodymium disc magnet with dimensions of 1″ diameter×0.25″ thickness. The magnetic field at its surface can be 2952 Gauss. Its magnetization direction can be axial (poles on the flat ends of the disc). It can weigh 24 grams. Magnets of other strengths and/or geometries (such as a cylinder or bar) can be used.

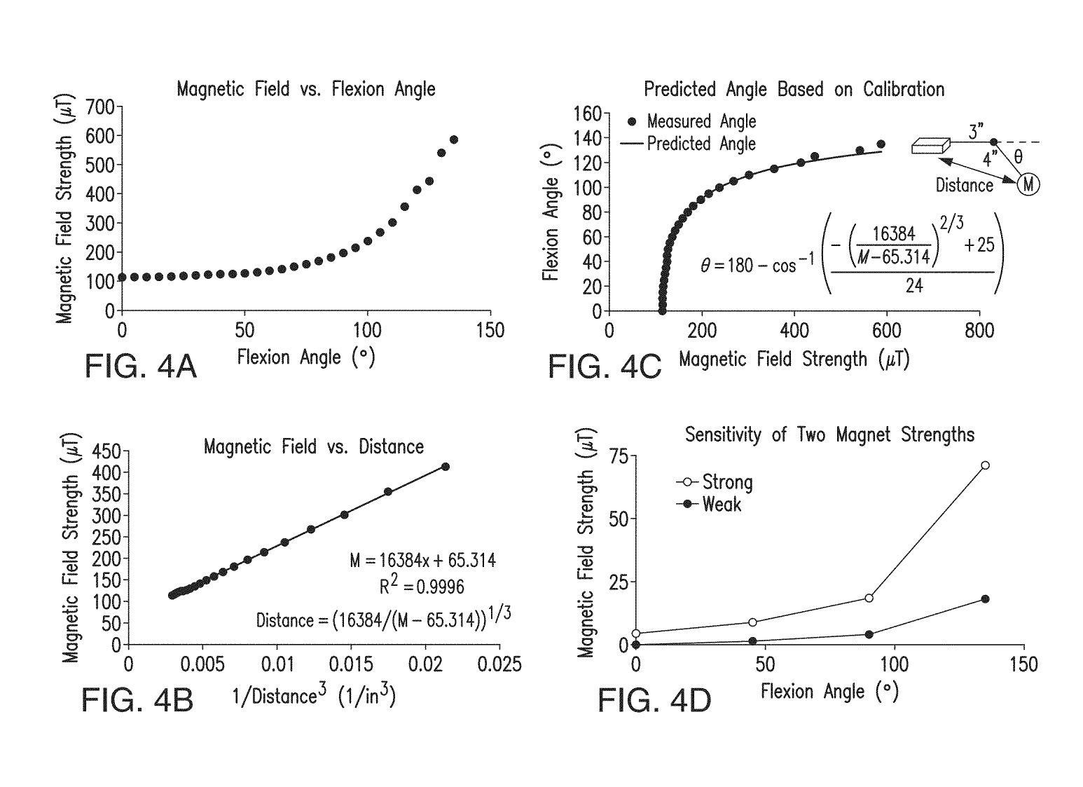

FIG. 4 shows, for the purpose of illustration and not limitation, data that can be used to calibrate the sensor. FIG. 4A shows a plot of magnetic field verse flexion angle. FIG. 4B shows a plot of magnetic field verse distance. FIG. 4C illustrates a plot of flexion angle verse magnetic field strength, which can provide a predicted angle based on the magnetic field strength measured. Inset in FIG. 4C illustrates how distance and flexion angle (θ) can be defined for the system described in FIGS. 4 A-C. “Flexion angle” as used in orthopaedics, and as used herein, is the supplementary angle of the angle between the sensor and the magnet. For example, the leg fully extended is given an angle of 0°, not 180°. The equations shown in FIGS. 4 A-C are specific to the configuration shown inset in FIG. 4C (i.e., dependent on sensor-joint and magnet-joint distances). Fig. D illustrates how the relationship between magnetic field strength and flexion angle is affected by magnet strength. A stronger magnet results in greater sensitivity to changes in distance (and thus angle). To measure changes in joint angle, the device and neodymium magnet can be fixed opposite from a hinge joint. The device can be kept stationary and the magnet moved across a range of flexion angles at 5° intervals. Position can be held for 5 seconds at each angle and the magnetic field strength can be recorded at 40 Hz. An equation relating sensor-magnet angle as a function of magnetic field magnitude can be derived to predict flexion angle. Flexion angles can be predicted by positioning a magnet opposite the sensor across a hinge joint, where the magnetic field increases exponentially with decreasing distance between the sensor and magnet. The calibration curve can be dependent on the sensor-to-joint and magnet-to-joint distances, as well as the magnet strength and magnetic pole orientation.

The magnetic field strength can be inversely proportional to the cube of the distance from the surface of the magnet. Thus, it is possible to relate magnetic field and distance between the sensor and the magnet. The flexion angle can be derived via the law of cosines, which allows one to calculate the third side of a triangle if one knows two sides and the angle between them, and to calculate the angles of a triangle if one knows the three sides. When the distance (A) between the sensor and the joint, the distance (B) between the magnet and the joint, and the angle (φ) between the sensor and the magnet are known (i.e., φ=180-θ), it is possible to calculate the distance between the sensor and the magnet. For example the distance (C) between the sensor and the magnet can be defined by equation 1:

C=\sqrt{A²+B²−2AB cos(φ)} (1)

When the distance (A) between the sensor and the joint, the distance (B) between the magnet and the joint, and the distance (C) between the sensor and the magnet are known, the angle (φ) between the magnet and the sensor can be defined by equation 2:

$$φ = arccos ( \frac{{A}^{2} + {B}^{2} - {C}^{2}}{2 AB} ) \qquad ( 2 )$$

To derive an equation predicting the flexion angle, the magnetic field strength at various flexion angles (example: 0, 30, 60, 90, and 120°) can be measured for a fixed sensor-joint and magnet-joint distance (FIG. 4A). Using the law of cosines, the sensor-magnet distance for each known angle can be calculated using Equation 1. The linear regression that relates the magnetic field as a function of the inverse of the distance cubed (1/distance³) (FIG. 4B) can be determined. Next, the distance can be calculated to determine what the sensor-magnet distance is for a given magnetic field, and the distance can be plugged it into Equation 2. This new equation allows one to calculate the flexion angle based on changes in the magnetic field strength (FIG. 4C). The relationship can allow one to solve for flexion angle for different distance and magnet combinations. Particular combinations would be sensitive enough to predict the angle at peak extension (far distance) without oversaturating the sensor at peak flexion (near distance), which can permit determination of the joint's full range of motion.

As an example and not by way of limitation, to measure changes in angle, the device and a neodymium magnet can be placed equidistant (6 inches) from a hinge joint. The device can be kept stationary and the magnet moved to flexion angles of 0, 45, 90, and 135° to simulate joint movement. Positions can be held for 5 s at each angle (n=3/group) and the magnetic field parallel to the magnetic dipole can be recorded. Significance can be assessed by two-way ANOVA with Bonferroni's post-hoc tests to compare magnetic field strength between groups (p<0.0001).

In a sample test, two magnets were tested: Weak (⅝″ diameter, ⅕″ thick) and Strong (1″ diameter, ¼″ thick). The sensor detected changes in magnetic field strength when a magnet was positioned at various angles relative to a pivot point. Magnetic field values were significantly different between all angles for both Weak and Strong magnets, with a power law relationship (p<0.0001). The Strong magnet induced higher magnetic fields at each angle and was more sensitive to changes in position than the Weak magnet (p<0.0001) (FIG. 4D).

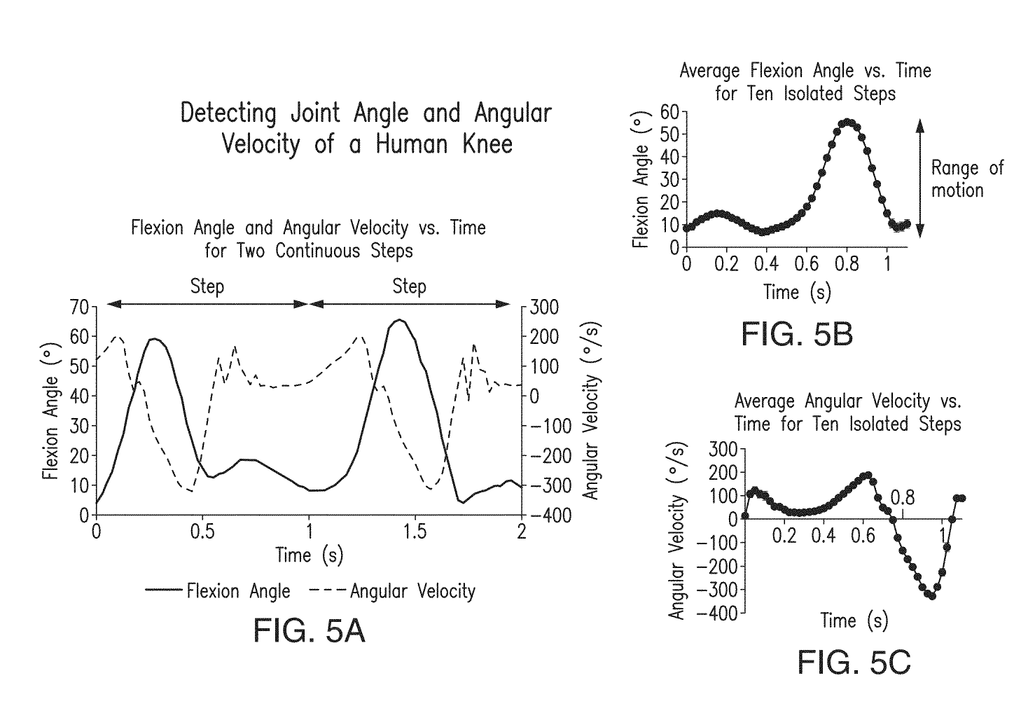

FIG. 5 shows, for the purpose of illustration and not limitation, exemplary data related to joint angle and angular velocity of a human knee. To capture dynamic range of motion of the human knee during normal gait, the sensor and magnet can be fixed distal and proximal to the knee joint on the posterior surface, respectively. The subject can walk at a step frequency of approximately 1 Hz, and the magnetic field strange and angular velocity of the tibia in the sagittal plane can be recorded at 40 Hz. Ten individual steps can be used to obtain the range of motion, defined as the difference between the minimum and maximum angles of the gait cycle. FIG. 5A shows the flexion angle and angular velocity verse time for two continuous steps of a human subject. The flexion angle and angular velocity of the tibia appear as repetitive and predictable patterns during ambulation. The dynamic range of motion of the knee in FIG. 5A is 54±4°, with a peak flexion angle of 55±3°. FIG. 5B shows the average flexion angle verse time for ten isolated steps. FIG. 5C shows the average angular velocity verse time for ten isolated steps. As described herein, the system is sufficient for gathering joint kinematics for a single leg. By wearing the system simultaneously on both legs (i.e., two systems), gait parameters can further be elucidated. In addition, the system can be used for other joints (for example, the elbow joint) and is not limited to the knee.

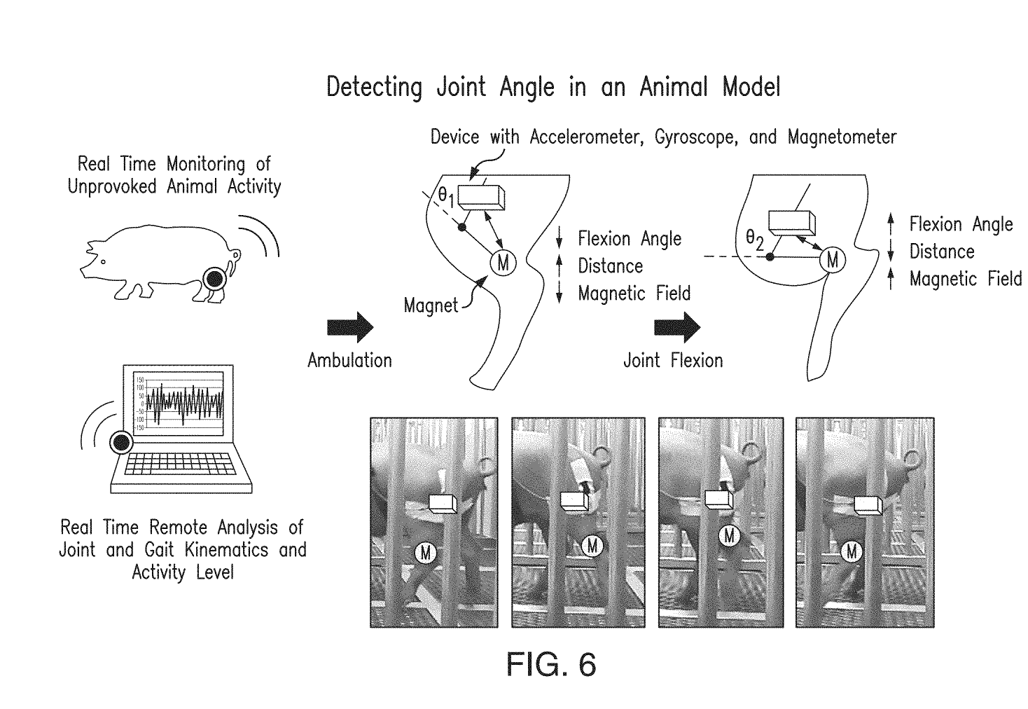

FIG. 6 shows, for the purpose of illustration and not limitation, an exemplary method for detecting joint angle in an animal model. The device can be attached to the femur and the magnet to the tibia of an animal. The device is described herein as attached to a pig; however, the method can be applied to any animal. The magnetic field can be measured as a function of flexion angle and an equation can be derived to relate changes in magnetic field to changes in flexion angle of the joint. High speed footage of a gait cycle is also shown, with device and magnet positions shown on the hind limb. The device can be attached proximal to the joint (above the joint). As in the human example discussed above, the configuration can be better suited for gathering gait data, since it is possible to obtain information about the acceleration and angular velocity of the tibia. Furthermore, the device and magnet can be incorporated into a brace, halter, clothing or other wearable article, to be worn by the user. The device and magnet can also be implanted into the animal. For example, the device can be inserted into the subcutaneous space, and a magnetic bone screw can be used in lieu of a traditional magnet.

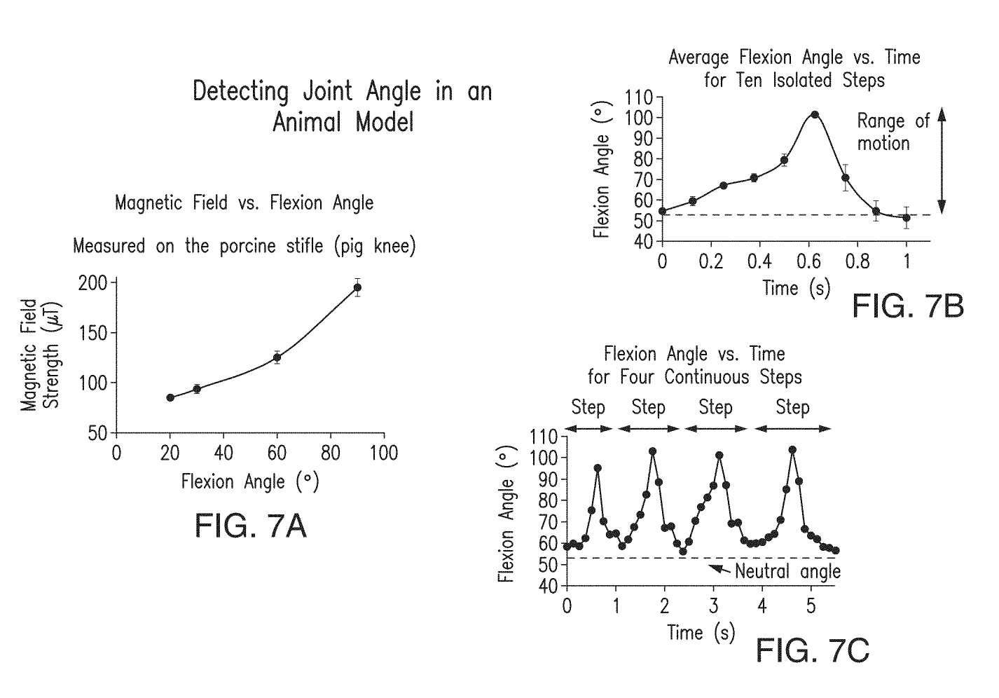

FIG. 7 shows, for the purpose of illustration and not limitation, exemplary data related to joint angle of a pig stifle joint (pig knee). FIG. 7A shows a plot of magnetic field verse flexion angle. FIG. 7B shows a plot of average flexion angle verse time for ten isolated steps. FIG. 7C shows flexion angle verse time for four continuous steps. In practice, the device and magnet can be attached to the hind limb proximal and distal to the stifle joint, respectively. The stifle can be manually flexed to angles of 20° (hyperextension), 30°, 60°, and 90°. The position can be held for 5 seconds at each angle and the magnetic field strength can be recorded at 8 Hz. An equation relating sensor-magnet angle as a function of magnetic field magnitude can be derived to predict flexion angle. The animal can be allowed to freely ambulate within a pen and the magnetic field strength can be recorded. Ten individual steps can be used to obtain the range of motion and can be visually confirmed with synchronized camera. The animal shown in FIG. 7 ambulated at a step frequency of approximately 1.25 Hz. The dynamic range of motion of the stifle during the gait cycle was 55±13°, with a peak flexion angle of 101±4°. Neutral stance flexion angle (the animal standing still) was measured as 53±13° and is indicated by the dashed line in FIGS. 7 B and C. These values are consistent with previously reported porcine range of motion.

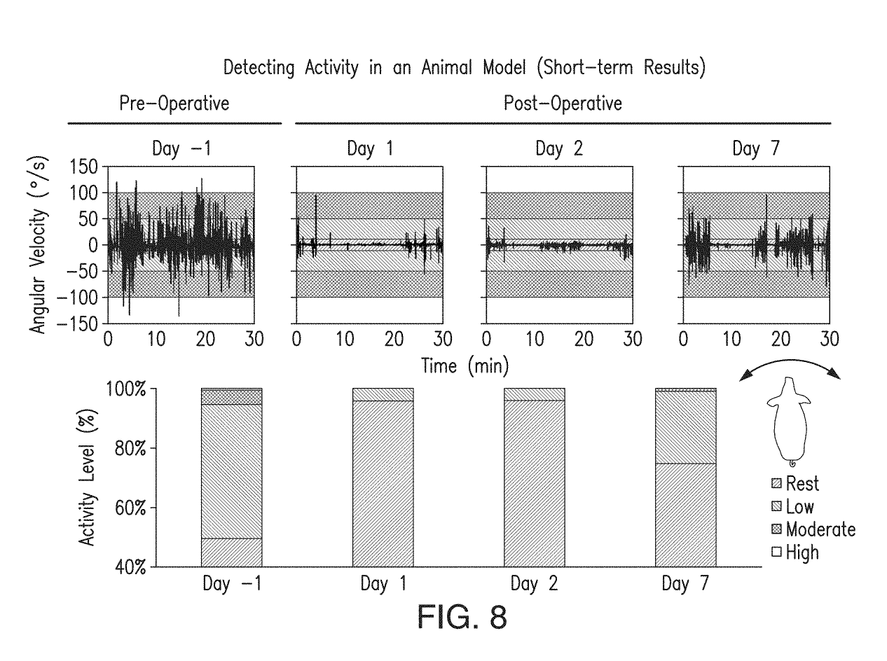

FIG. 8 shows, for the purpose of illustration and not limitation, example data from detecting short-term activity results in an animal model. In the example of FIG. 8, the device was attached to a harness worn by a castrated male Yucatan minipig (26 kg) pre- and post-surgery in an unrelated study involving bilateral arthrotomy of the stifle. Data was collected at 8 Hz for 30 minutes of unsupervised activity in a 4′×6′ pen on day −1 (pre-operative) and post-operative days 1, 2, and 7, with analgesics given for the first 5 days. Angular velocity (o/s) parallel to the dorsal plane (animal turning left or right) was recorded and the absolute values binned into four activity intensity levels: 0-5 (rest), 5-50 (low), 50-100 (moderate), and >100 (high).

On day −1 (pre-operative), the animal had full range of motion and baseline assessment was characterized by rest (49.6%) and low (45.1%) intensity activity, punctuated by short periods of moderate (4.9%) and high (0.4%) intensity activity. On days 1 and 2 (post-operative), the animal was predominantly sedentary (96% rest) and ambulated slowly with a stiff and limping gait (4% low intensity activity). By day 7, the animal had partially regained its baseline range of motion and activity level, such that low (24%) and moderate (0.9%) intensity activity accounted for a quarter of the test period.

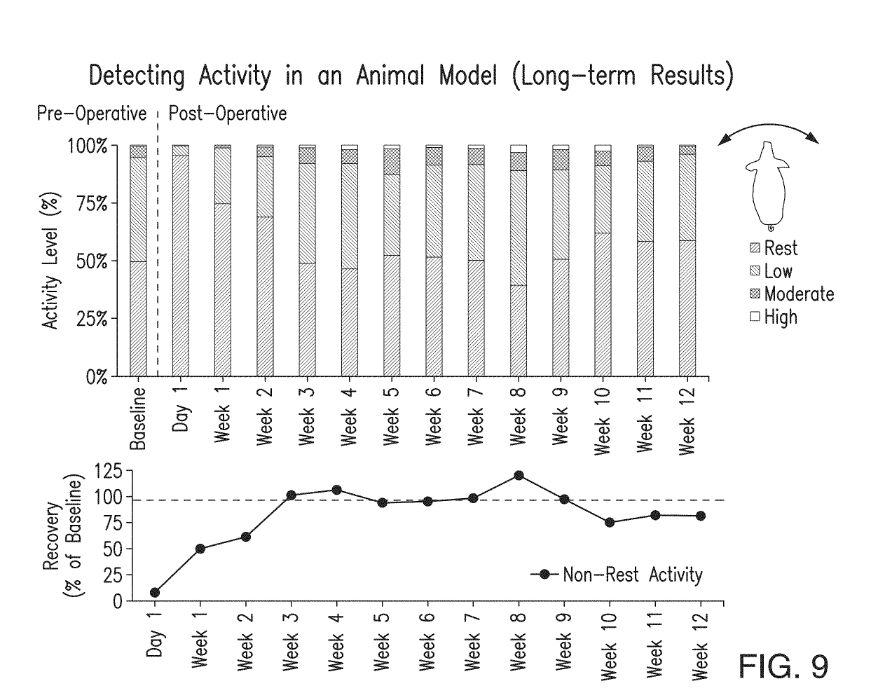

FIG. 9 shows, for the purpose of illustration and not limitation, example data from detecting long-term activity results in an animal model. In the example in FIG. 9, the device was attached to a harness worn by a castrated male Yucatan minipig (26 kg) pre- and post-surgery in an unrelated study involving bilateral arthrotomy of the stifle. Data was collected at 8 Hz for 30 minutes of unsupervised activity in a 4′×6′ pen pre-operatively on day −1 (baseline) and post-operatively on day 1 and weekly thereafter until euthanasia at week 12. Angular velocity (o/s) parallel to the dorsal plane (animal turning left or right) was recorded and the absolute values binned into four activity intensity levels: 0-5 (rest), 5-50 (low), 50-100 (moderate), and >100 (high).

On day −1 (baseline), the animal had full range of motion and activity was characterized by rest and low intensity activity, with short periods of moderate and high intensity activity. Immediately post-operative on day 1, the animal was primarily sedentary and ambulated with a stiff, limping gait. The animal had regained 50% of its pre-operative non-rest activity level by week 1, and was fully recovered by week 3. Non-rest activity levels were maintained until week 10, when it slightly decreased.

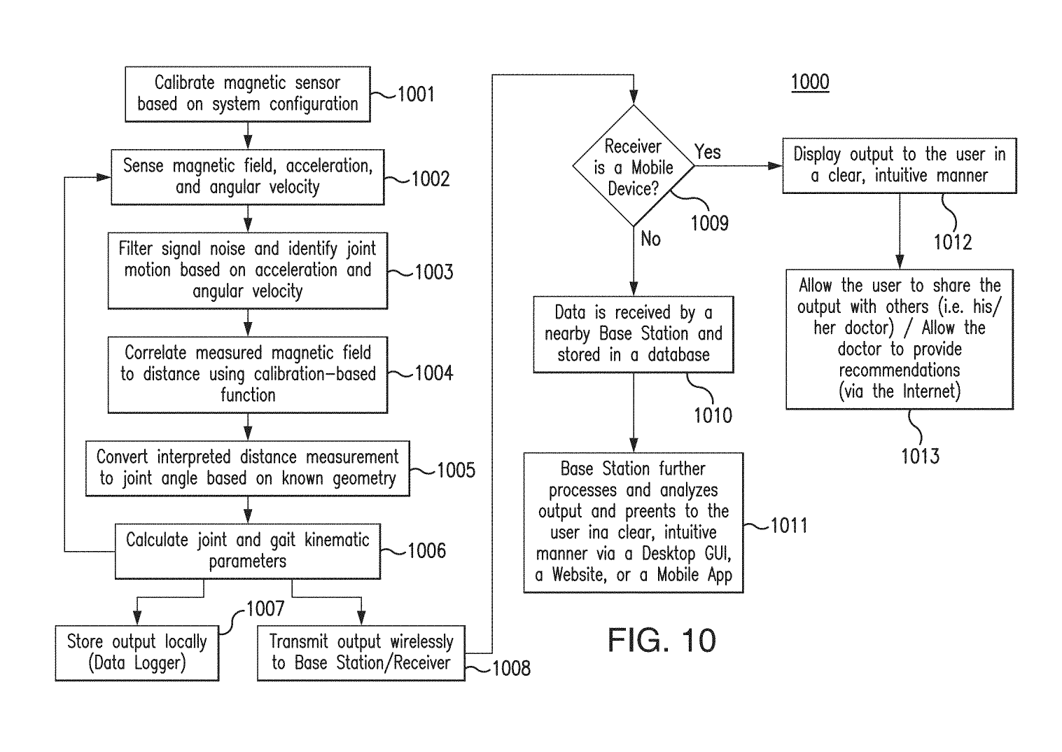

FIG. 10 shows, for the purpose of illustration and not limitation, a flow chart of an exemplary method (1000) of analyzing kinematics of a joint of an animal according to the disclosed subject matter. The method can include calibrating the magnetic sensor based on the exact configuration of the magnet and sensor at the time the device has been attached (1001). The calibration process can involve some sampling of the magnetic field while the joint is at prescribed angles. This can provide a function to be used for calculating a specific distance based on the measured magnetic field. The method can include using sensors on the sensor device to gather any relevant motion (1002). For example, the sensors can measure magnetic field, acceleration, and angular velocity. The signals can be filtered and motion parameters can be detected to identify joint motion (1003). For example, acceleration and angular velocity can be used to identify joint motion. The method can include correlating the measured magnetic field to a distance using the function obtained during step 1001 (1004). The distance measured can be used to calculate a joint angle (1005). This can be done given that the fixed magnet and sensor distances from the joint are known. The off-axis distance between the magnet and sensor can be calculated and trigonometry can be used to calculate the joint angle by knowing each side length of the triangle formed. The method can include calculating various joint and gait parameters (1006), such as angle of flexion and extension, angles of inward and outward rotation, stride length, swing time, stance time, gait symmetry, cadence, range of motion, and others. Data can be stored locally (1007) and/or transmitted wirelessly (1008), for example, to be presented to the user via a user interface. In some instances, the level of processing necessary for steps 1000-1006 can be larger than possible on a small, wearable device. In those cases, the unprocessed, raw data can be stored locally (1007) and/or transmitted wirelessly to a receiver (1008) where additional processing and analysis can be performed.

After the joint and gait parameters are calculated, the output can be simultaneously stored locally (1007) (for example, for backup purposes) and/or transmitted wirelessly (1008) to another system where it can be displayed to the user in an intuitive manner (e.g., charts, scores, recommendations or other related formats), or it can be further processed and analyzed. If the receiver is a mobile device (1009), the output can be presented on a mobile application interface (1012). The user can be allowed to share the output with others or the doctor can be allowed to provide recommendations (for example, via the internet) (1013). If the receiver is not a mobile device (1009), the data can be stored in a database of a base station (1010) and the output can be presented via a desktop graphical user interface, a website, or transmitted to a mobile application interface (however without a direct link between the wearable device and the mobile device).

In an exemplary test, data from a wearable device as disclosed herein was compared with data acquired from a motion capture system to confirm the range of motion measurements (ROM) during normal human gait. The system was then used to track the recovery of porcine subjects after bilateral arthrotomy to investigate alterations in physical activity and gait over time.

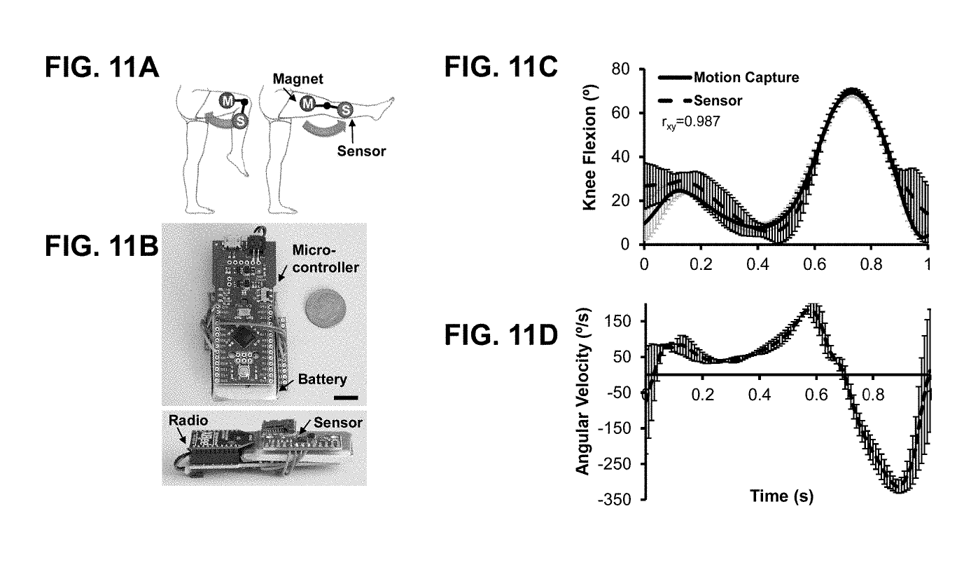

With reference to FIG. 11 for the purpose of illustration and not limitation, the device consisted of a microcontroller, radio, data logger, battery and sensor (triple-axis accelerometer, gyroscope, and magnetometer) within a plastic enclosure (FIG. 11B). To measure joint angle, a neodymium magnet (1″×½″×¼″) and the device were affixed 3″ from the knee on the lateral femur and tibia, respectively (FIG. 11A). The knee was flexed to increasing angles (0°, 30°, 45°, and 60°) and an equation relating joint angle to the magnetic field magnitude was derived. To confirm that the device could capture dynamic ROM of the knee, healthy human subjects (n=3) walked at a step frequency of ˜1 Hz, and the magnetic field strength and angular velocity were recorded at 100 Hz. A 12-camera motion capture system (Raptor Series, Motion Analysis Corp.) was used concurrently to detect knee flexion using 3D trajectories of markers attached to the leg (Visual3D, C-Motion). Knee flexion angle was calculated for discrete steps (n=10-32/subject) to determine the average ROM during the gait cycle. A correlation coefficient (r_{xy}) was computed using MATLAB to compare the two methods.

To evaluate physical activity of porcine subjects, the device was attached to a harness worn by castrated male Yucatan minipigs pre- and post-surgery (n=4) in an unrelated study involving bilateral arthrotomy of the stifle, with analgesics given for the first 5 days after surgery. Data was collected at 40 Hz for 30 minutes of unsupervised activity in two connected 4′×6′ pens pre-operatively (Baseline) and post-operatively on Day 1 and bi-weekly thereafter until euthanasia at Week 10. Animals were considered active when the angular velocity parallel to the dorsal plane (animal turning left or right) was >5%/s. To quantify longitudinal changes in joint kinematics (n=1), the device and magnet were affixed to the stifle as previously described and the animal was allowed to freely ambulate in the pen. Data was collected pre-operatively (Baseline) and bi-weekly post-operative. Discrete steps were identified by local maxima in the magnetic field (n=8-15/time point) and used to determine the angular velocity of the tibia during the gait cycle. Significance was assessed by student's t-test and one-way ANOVA with Tukey's post-hoc tests to compare between groups (p<0.05).

The exemplary test established a wearable device capable of quantifying joint kinematics in humans and translatable to a large animal model to monitor joint function. Flexion angle was predicted via changes in the magnetic field strength, which increased with flexion. Knee flexion measurements during normal gait cycles closely correlated with data acquired by the motion capture system (FIG. 11C, r_{xy}=0.987). The device also detected the characteristic pattern of the tibial angular velocity during the gait cycle (FIG. 11D). The average ROM (68.8±1.4° vs. 66.4±3.7°) and peak flexion angle (71.0±0.4° vs. 69.2±1.7°) were similar between the wearable device and the motion capture system (p>0.05).

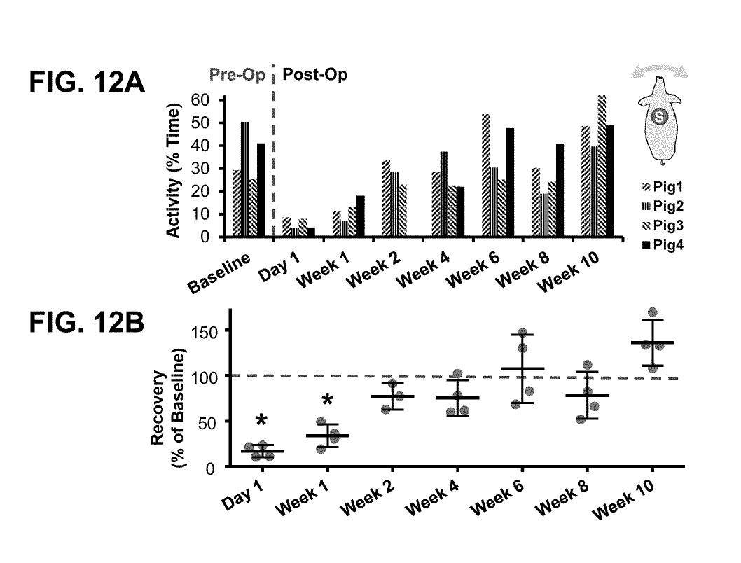

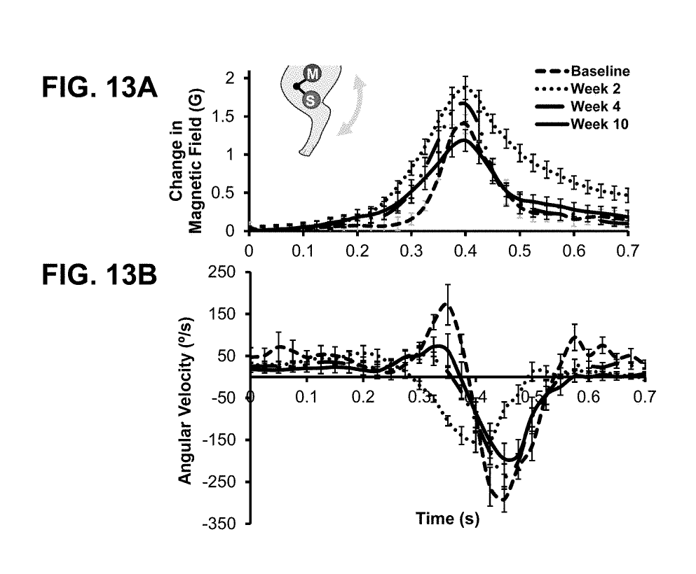

The device was used to monitor unsupervised animal activity and joint kinematics pre- and post-arthrotomy over 10 weeks (FIG. 12A). Activity was reduced to 17% of the pre-operative level (Baseline) on Day 1 after surgery (FIG. 12B). By Week 1, the animals had regained 34% of Baseline activity, and by Week 2, there was no significant difference compared to Baseline. Activity levels increased with time and remained at the Baseline level until Week 10. To evaluate stifle kinematics over time, the device and magnet were worn on the hindlimb of one animal and discrete steps were identified from magnetic field maxima (FIG. 13A). Altered joint motion was most apparent at Week 2, where the angular velocity of the tibia was reduced during the swing phase, indicating a stiffer gait (FIG. 13B). Although the angular velocity increased with time to approach the Baseline level, this abnormality was still detectable at Week 10, suggesting incomplete recovery.

Motion sensors can provide objective data for musculoskeletal research, especially for large animal models where pain and functional outcomes can be difficult to measure. To that end, the wearable device disclosed herein can accurately measure both joint kinematics and activity using a single integrated sensor. By placing a magnet opposite the articulating joint, the device can detect steps, measure joint ROM, and assess limb angular velocity in an unsupervised setting, facilitating the longitudinal assessment of research subjects. As described hereinabove, a porcine cohort was monitored after arthrotomy and the joint activity monitoring system described herein found that return to pre-operative activity occurred approximately 2 weeks after surgery. The joint kinematics recovered slower than activity level, suggesting that joint kinematics is a more sensitive measure of functional recovery.

In some embodiments, a machine learning algorithm can be used to classify gait and activity patterns, as well as correlate joint function to the structure and function of intra-articular tissues.

The foregoing merely illustrates the principles of the disclosed subject matter. Various modification and alterations to the described embodiments will be apparent to those skilled in the art in view of the teachings herein. It will thus be appreciated that those skilled in the art will be able to devise numerous techniques which, although not explicitly described herein, embody the principles of the disclosed subject matter and are thus within the spirit and scope.

1. A joint analysis system for analyzing kinematics of an anatomical joint, the joint having a first side and a second side, the joint analysis system comprising:
a sensor device, configured to be disposed on the first side of the joint, the sensor device having:
one or more sensors;
a processor coupled to the one or more sensors;
a wireless data transmitter coupled to the processor;
a data storage device coupled to the processor; and
a battery coupled to the sensors, processor, wireless data transmitter, and data storage device;
a magnet, configured to be disposed on the second side of the joint; and
an analysis engine, configured to receive data from the sensors.

2. The system of claim 1, wherein the one or more sensors includes a magnetometer.

3. The system of claim 2, wherein magnetometer sensor is adapted to provide readings that are influenced by the magnetic field provided by the magnet to provide kinematic information of the joint.

4. The system of claim 1, wherein the one or more sensors includes an accelerometer.

5. The system of claim 1, wherein the one or more sensors includes a gyroscope.

6. The system of claim 1, wherein the one or more sensors are further configured to sense stride length.

7. The system of claim 1, wherein the one or more sensors are further configured to sense swing time.

8. The system of claim 1, wherein the one or more sensors are further configured to sense stance time.

9. The system of claim 1, wherein the one or more sensors are further configured to sense ambulation speed.

10. The system of claim 1, wherein the one or more sensors are further configured to sense distance traveled.

11. The system of claim 1, wherein the one or more sensors are further configured to sense gait symmetry.

12. The system of claim 1, wherein the one or more sensors are further configured to sense gait cadence.

13. The system of claim 1, wherein the one or more sensors are further configured to sense joint kinematics.

14. The system of claim 1, wherein the one or more sensors are further configured to sense a disrupted pattern of ambulation.

15. The system of claim 1, wherein the data analysis engine is further configured to recognize an abnormal gait or behavior.

16. The system of claim 1, further comprising a base station, the base station comprising
a processor;
a data storage device coupled to the processor;
a user interface coupled to the processor; and
a wireless data transmitter coupled to the processor and configured to communicate with the wireless data transmitter of the sensor device.

17. The system of claim 16, wherein the base station further comprises a display.

18. The system of claim 1, wherein the sensor device and the magnet are configured to be worn by an animal or human.

19. The system of claim 1, wherein the sensor device and the magnetic are configured to be implanted in an animal or human.

20. A method of analyzing kinematics of an anatomical joint, comprising:
calibrating one or more sensors and a magnet relative the joint;
sensing a magnetic field with the calibrated sensors while the joint exhibits motion and angular velocity to generate a signal;
filtering signal noise, if any, from the signal and identifying joint motion based on the corresponding acceleration and angular velocity; and
calculating joint and gait kinematic parameters from the identified joint motion.
